(19) United States
(12) Patent Application Publication
(10) Pub. No.: US 2023/0228765 A1
(43) Pub. Date: Jul. 20, 2023

(54) METHODS AND ASSOCIATED USES, KITS AND SYSTEM FOR ASSESSING SEPSIS

(71) Applicant: The Secretary of State for Defence, Salisbury, Wiltshire (GB)

(72) Inventors: Thomas Robert Laws, Salisbury (GB); Roman Antoni Lukaszewski, Salisbury (GB)

(73) Assignee: The Secretary of State for Defence, Salisbury, Wiltshire (GB)

(21) Appl. No.: 18/001,291

(22) PCT Filed: Jun. 17, 2021
(86) PCT No.: PCT/GB2021/000074

§ 371 Date: Dec. 9, 2022

(30) Foreign Application Priority Data
Jun. 19, 2020 (GB) 2009401.7

(51) Int. Cl. G01N 33/68

(52) U.S. Cl. CPC G01N 33/6893; G01N 2800/26; G01N 2800/52

(57) ABSTRACT

The invention relates to protein biomarkers representing protein biomarker signatures to assess a patient who may develop sepsis, or who may have developed sepsis. The invention relates in particular to methods for assessment or monitoring with respect to diagnosis, prediction or progression of sepsis in a patient, as well as the responsiveness to, or selection of suitable agents for, the treatment of sepsis. The invention also relates to the use of protein biomarkers representing protein biomarker signatures for sepsis, and associated kits and system.

### TECHNICAL FIELD OF THE INVENTION

The invention relates to protein biomarkers representing protein biomarker signatures to assess a patient who may develop sepsis, or who may have developed sepsis. The invention relates in particular to methods to assess whether a patient may develop sepsis or to diagnose a patient as having sepsis, monitoring a patient to predict whether and when the patient may develop sepsis, or to monitor the progression of sepsis in the patient, monitoring the responsiveness of a patient to treatment with an antimicrobial agent(s) and/or immunosuppressive agent(s), or selecting a therapeutic agent(s) and/or immunosuppressive agent(s) for administration to a patient predicted or diagnosed as having sepsis. The invention also relates to use of protein biomarkers representing protein biomarker signatures for sepsis, and kits and systems for assessing or monitoring a patient to predict or diagnose sepsis, the response of the patient to treatment for sepsis, or selecting a therapeutic agent(s) and/or immunosuppressive agent(s) for treatment of sepsis.

### BACKGROUND TO THE INVENTION

Sepsis is a significant worldwide concern, reflected by this pathological syndrome considered the primary cause of death in patients from infection and costing the healthcare sector many tens of billion U.S. dollars per year. In the UK, the economic impact of sepsis to the National Health Service is estimated as a direct cost of £2 billion per year, in addition to indirect costs of up to £13.6 billion. During the COVID-19 pandemic that spread globally during 2019-20, sepsis was confirmed as a key clinical presentation in patients seriously infected with SARS-CoV-2.

From a clinical perspective, sepsis is a challenging condition to resolve on account of factors including: the often rapid onset of disease requiring a similarly timely diagnosis and/or administration of appropriate medical intervention protocols; a lack of a validated, standard diagnostic test; the variety of clinical presentation(s) often complicating diagnosis; the range of infectious agents capable of causing sepsis; and the difficulty in identifying the infectious agent in question, increasing the likelihood of an initial wide-spectrum antimicrobial agent(s) selection not providing optimal or effective treatment. As patient symptoms can initially present as non-specific to sepsis, there is the potential for clinicians to administer treatment with incorrect or non-optimal antimicrobial regimens, risking contributing to the on-going antimicrobial resistance crisis. Indeed, once symptoms appear, there is an inverse correlation between effectiveness of treatment and patient outcome.

The medical definition of sepsis has developed over the years. Sepsis was initially defined in 1991 as a host's Systemic Inflammatory Response Syndrome (SIRS) to infection (‘Sepsis-1’), identified by clinical parameters based on two or more of temperature level, heat rate, respiratory rate or white blood cell count. More significant incidences, which included organ failure, were considered severe sepsis. Revision of the sepsis and severe sepsis definitions in 2001 were based on the inclusion of further clinical parameters, which may evidence infection in a host (‘Sepsis-2’). However, greater understanding of sepsis led healthcare specialists in 2014-15 to provide an updated definition of sepsis as a ‘life-threatening organ dysfunction caused by a dysregulated host response to infection’ (Singer et al. 2016. The Third International Consensus Definitions for Sepsis and Septic Shock (‘Sepsis-3’). JAMA; 315(8): 801-810). Supporting diagnostic criteria for the Sepsis-3 definition included changes in a subject's Sequential Organ Failure Assessment (SOFA) score, used by medics to predict the outcome of critically-ill patients and based on parameters associated with the respiratory, nervous and cardiovascular systems, as well as functionality of the liver, coagulation and kidneys. Use of a quickSOFA (qSOFA) score, which applies diagnostic criteria encompassing respiratory rate, altered mentation and systolic blood pressure, was incorporated into the Sepsis-3 definition. Accordingly, septic shock was defined as ‘a subset of sepsis in which underlying circulatory and cellular/metabolic abnormalities are profound enough to substantially increase mortality’.

Efforts to standardise the definition of sepsis provides greater clarity for clinicians when establishing, and initiating treatment against, incidences of sepsis. However, there remains an urgent need for a reliable, rapid, simple-to-operate, point-of-care test validated against, and capable of early diagnosis of, sepsis, in particular sepsis falling within the Sepsis-3 definition that requires life-threatening organ dysfunction.

### SUMMARY OF THE INVENTION

According to a first aspect, the invention provides a method for analysing a biological sample, obtained from a patient, to assess whether the patient may develop sepsis or to diagnose the patient as having sepsis, the method comprising the steps of:
a. determining in the biological sample individual levels of protein biomarkers representing a protein biomarker signature; and
b. using the individual levels of the protein biomarkers collectively to assess whether the patient may develop sepsis or to diagnose a patient as having sepsis,

wherein the protein biomarkers of the protein biomarker signature comprises at least four biomarkers from a list consisting of CCL-16, CD28, CD244, FGF21, GALNT3, GT, IL-18BP, JAM-A, LDL-R, LILRB5, LTBR, MCP-2, MMP-1, NUCB2, SIGLEC10, TNF-R1, TNF-R2, TNFRSF10A, TNFRSF11A, TNFRSF14, TRAILR2 and UPAR.

The term ‘biological sample’ includes, but not exclusively, blood, serum, plasma, urine, saliva, cerebrospinal fluid or any other form of material, preferably fluid-based or capable of being converted into a fluid-like state (e.g. tissue which can be broken down or separated in a solution, such as a buffered solution), which can be extracted or collected from a patient.

The term ‘sepsis’ is understood to refer to sepsis in accordance with the Sepsis-3 definition described above.

Previous studies have identified host immune responses, represented by a biomarker signature, which can be used to provide early diagnosis of sepsis. However, such studies commonly focus on gene-based biomarker signatures, reflecting the high sensitivities of nucleic-acid-based detection methods, and often describe relatively large numbers of such biomarkers (e.g. 15-40 gene targets) due to the relative ease in concurrently multiplexing techniques such as Polymerase Chain Reaction (PCR).

Furthermore, such historic studies were conducted using previous sepsis definitions and hence would not be compatible for Sepsis-3, which requires organ dysfunction as one of the key disease complications.

The Applicant has identified, through a comprehensive analysis of carefully characterised host samples (i.e. characterised according to the Sepsis-3 definition), a 22-protein panel of biomarkers highly significant to predicting sepsis i.e. an ability to predict infection and organ dysfunction. From this 22-protein panel, the Applicant has identified a series of protein combinations that represent biomarker signatures capable of predicting sepsis, offering mean Area Under The Curve (AUC) values greater than 0.72, with one exemplified protein biomarker signature having an AUC of 0.86 at Day −1 prior to clinical diagnosis of sepsis. Thus, such biomarker signatures can offer a high level of confidence for pre-condition diagnosis for sepsis involving organ dysfunction as a consequence of infection and overwhelming immune dysregulation. These biomarker signatures also offer a mean AUC of greater than 0.72 for providing confirmatory diagnosis of sepsis, with one exemplified protein biomarker signature having an AUC of 0.87 i.e. a high level of confidence for biomarker signatures providing a confirmatory diagnosis. The subsets identified by the Applicant offer a manageable number of protein targets in a protein biomarker signature, e.g. having 4-proteins, thus being suitable for transitioning onto current protein diagnostic platform technologies.

The 22 proteins identified are summarised below in Table 1. Information regarding each protein can be found at www.uniprot.org/ (see ‘Uniprot reference’ column, which provides the corresponding Uniprot Accession Number reference for each protein, which enables access to information including each protein's sequence).

TABLE 1
A summary of the 22 proteins found to be highly significant in predicting Sepsis-3

| Protein | Reference | Uniprot reference |
|---|---|---|
| C-C motif chemokine 16 | CCL16 | https://www.uniprot.org/uniprot/O15467 |
| T-cell-specific surface glycoprotein CD28 | CD28 | https://www.uniprot.org/uniprot/P10747 |
| Natural killer cell receptor 2B4 | CD244 | https://www.uniprot.org/uniprot/Q9BZW8 |
| Fibroblast growth factor 21 | FGF21 | https://www.uniprot.org/uniprot/Q9NSA1 |
| Polypeptide N- | GALNT3 | https://www.uniprot.org/uniprot/Q14435 |
| acetylgalactosaminyltransferase 3 | | |
| Gastrotropin | GT | https://www.uniprot.org/uniprot/P51161 |
| lnterleukin-18-binding protein | IL-18BP | https://www.uniprot.org/uniprot/O95998 |
| Junctional adhesion molecule A | JAM-A | https://www.uniprot.org/uniprot/Q9Y624 |
| Low-density lipoprotein receptor | LDL-R | https://www.uniprot.org/uniprot/P01130 |
| Leukocyte immunoglobulin-like | LILRB5 | https://www.uniprot.org/uniprot/O75023 |
| receptor subfamily B member 5 | | |
| Lymphotoxin-beta receptor | LTBR | https://www.uniprot.org/uniprot/P36941 |
| Monocyte chemotactic protein 2 | MCP-2 | https://www.uniprot.org/uniprot/P80075 |
| Matrix metalloproteinase-1 | MMP-1 | https://www.uniprot.org/uniprot/P03956 |
| Nucleobindin-2 | NUCB2 | https://www.uniprot.org/uniprot/P80303 |
| Sialic acid-binding Ig-like lectin 10 | SIGLEC10 | https://www.uniprot.org/uniprot/Q96LC7 |
| Tumor necrosis factor receptor 1 | TNF-R1 | https://www.uniprot.org/uniprot/P19438 |
| Tumor necrosis factor receptor 2 | TNF-R2 | https://www.uniprot.org/uniprot/P20333 |
| Tumor necrosis factor receptor | TNFRSF10A | https://www.uniprot.org/uniprot/O00220 |
| superfamily member 10A | | |
| Tumor necrosis factor receptor | TNFRSF11A | https://www.uniprot.org/uniprot/Q9Y6Q6 |
| superfamily member 11A | | |
| Tumor necrosis factor receptor | TNFRSF14 | https://www.uniprot.org/uniprot/Q92956 |
| superfamily member 14 | | |
| TNF-related apoptosis-inducing | TRAIL-R2 | https://www.uniprot.org/uniprot/O14763 |
| ligand receptor 2 | | |
| Urokinase plasminogen activator | U-PAR | https://www.uniprot.org/uniprot/Q03405 |
| surface receptor | | |

Assuring confidence in the classification of patient condition is a key task for any study that is reliant on clinical opinion to baseline data used in subsequent analytical techniques. Any errors of clinical judgement in identifying sepsis in the study cohort is likely to have a substantial impact on the performance of statistical models produced following analysis of patient samples. A key advantage of the clinical study underpinning the Applicant's research is the involvement of clinical experts in the field of sepsis, who have retrospectively reviewed patient data to agree on an accurate day of diagnosis according to the Sepsis-3 definition.

The Applicant conducted studies that measured 718 protein analytes in subject samples obtained from the wide-ranging clinical study that carefully categorised each patient according to either a) diagnosis of sepsis according to the Sepsis-3 definition, b) control (i.e. no specific pathology) or c) Systemic Inflammatory Response Syndrome (‘SIRS’ i.e. a non-specific inflammation response in a host without an adjudication of infection). Both the control and SIRS samples were used as comparator controls in the identification of sepsis-relevant biomarkers. Comparative statistical analysis of the protein abundance between comparators and patients who went on to develop sepsis led to the identification of small subsets e.g. combinations of between two and ten protein biomarkers, in particular between four and ten proteins, from a list of 22 identified proteins, which provide a high diagnostic capacity to estimate a patient's risk of developing sepsis.

The statistical analysis for identifying protein biomarker sets comprised the following steps. Firstly, all proteins that had little or no differential abundance across the three sample categories (sepsis; control; SIRS) were eliminated, with remaining proteins taken on to the next steps in the analysis. This elimination was achieved by setting upper and lower thresholds of abundance for the control patient's proteins. The numbers of sepsis patients (samples) with abundances for the same proteins being outside of these upper and lower limits were then deemed more likely to have predictive potential, and thus retained in the analysis, with all other proteins (having a similar abundance between the upper and lower limit) being disregarded. Secondly, a further down-select was applied to find a manageable numbers of proteins for a diagnostic system. Extensive testing of every combination of the down-selected protein analytes presents a very computationally difficult and complex task. One approach might be to find the protein analytes that are most different between the desired population and the control populations. In the Applicant's opinion, this was a flawed approach for the data in question because this is a mixed population of sepsis subjects. The following purely hypothetical example can be used to explain how this down-select was conducted. If one considers pneumococcal infection as the most prevalent form of disease for the sepsis patient cohort and viral sepsis the second most prevalent, it can be assumed that these conditions might have different immunological profiles. If this were to be the case, then picking only the best ‘hits’ will make a diagnostic for pneumococcal infection and would not achieve a goal of a pan-sepsis diagnostic i.e. a diagnostic capable of diagnosing sepsis resulting from infection by different infectious agents (e.g. sepsis resulting from bacterial infection, sepsis resulting from viral infection etc.). The Applicant's approach instead was to find the protein analytes whose abundance was least related within the down-selected list, to select the diagnostic proteins for all prevalent forms of Sepsis-3. These smaller subsets were evaluated for predictive potential using neurological networks.

The Applicant's approach differs from traditional down-selection methods where a hypothesis test is used and only statistically significant proteins are included. The rational for deviating from these methods is that the patient population has substantial heterogeneity and are not made of defined groups. For example, sepsis will include a variety of primary loci of infection and disease (i.e. pneumonia, meningitis etc.) and potentially a variety of different pathogens, whereas the SIRs may be a result of “sterile inflammation” or even auto-immunity. This can mean that the data can take bimodal forms where some sepsis individuals have altered abundance and some not. This ‘hidden’ aspect of the data prevents accurate hypothesis testing. A second reason to deviate from hypothesis tests is that the Applicant has used a very accurate assay system for estimating relative concentrations of proteins across populations.

From the 22-biomarker protein panel identified, 22 specific biomarker signatures (summarised in Table 3; biomarker signatures A-V) were elucidated that provide combinations of from 2 to 10 biomarkers capable of pre-condition diagnosis (denoted as Day −1 i.e. one day before sepsis diagnosis), and confirmatory diagnosis (denoted as Day 0 i.e. day of sepsis diagnosis), of sepsis in subjects. In particular, 20 biomarker signatures comprising at least four biomarkers from the 22-protein list (biomarker signatures A-G, and J-V) were identified for incorporation in the methods of the invention, and the corresponding uses, kits and systems, as described as follows.

Preferably, the protein biomarkers of the protein biomarker signature comprises at least four biomarkers, at least five biomarkers, at least six biomarkers, at least seven biomarkers, at least eight biomarkers, at least nine biomarkers or at least ten biomarkers from a list consisting of CCL-16, CD28, CD244, FGF21, GALNT3, GT, IL-18BP, JAM-A, LDL-R, LILRB5, LTBR, MCP-2, MMP-1, NUCB2, SIGLEC10, TNF-R1, TNF-R2, TNFRSF10A, TNFRSF11A, TNFRSF14, TRAILR2 and UPAR Preferably, the protein biomarker signature comprises CCL-16 and MCP-2. These biomarkers were shown to be common to 15 of the 20 preferred biomarker signatures (A-B, J-V). Furthermore, these biomarkers are included in those biomarker signatures shown to have the highest performance with respect to predicative efficacy (biomarker signature N (mean AUC of 0.86 for both Days −1 and 0 respectively); biomarker signature A (mean AUC of 0.80 and 0.86 at Days −1 and 0 respectively)). Indeed, all biomarker signatures had an AUC of greater than 0.81 at Day 0.

Preferably, the protein biomarker signature comprises or consists of LTBR, CCL16, CD28, FGF21 and MCP-2. The specific 5-protein biomarker signature (J) offered a mean AUC of 0.76 at Day −1 and 0.87 at Day 0, thus in particular providing the strongest confirmatory diagnosis of sepsis while using a relatively small number of proteins, thus being especially advantageous in terms of being suitable for integration onto a diagnostic protein platform.

Alternatively, the protein biomarker signature further comprises GALNT3, GT, LDL-R, LILRB5 and MMP-1. This biomarker combination i.e. also comprising at least CCL-16 and MCP-2, represented 14 of the 20 identified biomarker signatures (including biomarker signatures A and N). All mean AUCs for these biomarker signatures were greater than 0.81 at Day 0, again offering a positive confirmatory diagnosis of sepsis.

Preferably, the protein biomarker signature further comprises FGF21. This biomarker combination i.e. also comprising at least CCL-16, MCP-2, GALNT3, GT, LDL-R, LILRB5 and MMP-1 represented 13 of the 20 identified biomarker signatures (including biomarker signatures A and N), supporting the view that a key subset of biomarkers exists from the 22-identified proteins that can form the basis for pre-condition/confirmatory diagnosis of sepsis.

Preferably, the protein biomarker signature comprises or consists of CCL16, CD28, FGF21, GALNT3, GT, LDL-R, LILRB5, MCP-2, MMP-1 and TNFRSF11A. This particular biomarker signature (N) provided the highest predictive efficacy at Day −1 across the 20 biomarker subsets with a mean AUC of 0.86, and the joint-second highest performing mean AUC at Day 0 (0.86). This biomarker signature therefore represents a particularly attractive option in terms of pre-condition and/or confirmatory diagnosis of Sepsis-3.

Preferably, the protein biomarker signature comprises or consists of CCL16, CD244, FGF21, GALNT3, GT, LDL-R, LILRB5, MCP-2, MMP-1 and TNF-R1. This particular biomarker signature (K) provided the second highest predictive efficacy at Day −1, with a mean AUC of 0.81. Furthermore, this biomarker signature provided a mean AUC of 0.87 at Day 0.

Preferably, the protein biomarker signature comprises or consists of CCL16, CD28, FGF21, GALNT3, GT, LDL-R, LILRB5, MCP-2, MMP-1 and TNF-R1. This particular biomarker signature (A) provided high performing mean AUCs at Days −1 and 0, reporting values of 0.80 and 0.86 respectively.

Preferably, the protein biomarker signature comprises or consists of CCL16, CD28, FGF21, GALNT3, GT, LDL-R, LILRB5, MCP-2, MMP-1 and U-PAR. In the same manner of biomarker signature A, this particular biomarker signature (V) also provided mean AUCs at Days −1 and 0 of 0.80 and 0.86 respectively.

Preferably, the protein biomarker signature comprises or consists of CCL16, CD28, FGF21, GALNT3, GT, LDL-R, LILRB5, MCP-2, MMP-1 and TRAIL-R2. This particular biomarker signature (M) provided mean AUCs at Days −1 and 0, reporting values of 0.78 and 0.85 respectively.

Preferably, the protein biomarker signature further comprises at least one additional biomarker taken from a list of biomarkers categorised as pro-inflammatory cytokines, anti-inflammatory cytokines, chemokines, acute phase reactants, cell receptors/mediators or vascular markers.

Preferably, the protein biomarker signature further comprises at least one additional biomarker from a list consisting of procalcitonin (PCT), lactate, C-reactive protein (CRP), D-Dimer and pancreatic stone protein (PSP).

According to a second aspect, the invention provides a method for analysing biological samples, obtained from a patient at risk of, or having developed, sepsis, to monitor the patient, the method comprising the steps of:
a. determining in the biological samples, obtained from the patient at a plurality of time points, individual levels of protein biomarkers representing a protein biomarker signature, wherein the protein biomarker signature comprises the biomarkers selected according to the first aspect; and
b. using changes in the individual levels of the protein biomarkers collectively, across the plurality of time points, to monitor the patient and to predict whether the patient may develop sepsis, or to monitor the progression of sepsis in the patient.

This aspect is particularly beneficial in identifying patients whose condition with respect to sepsis, i.e. infection and organ dysfunction, is worsening or indeed improving. For example, observing continued/increasing changes in the biomarkers comprising the biomarker signature likely indicates that a patient is still, or is increasingly, septic, suffering organ dysfunction and/or hosting an infective agent as reflected by continued host biomarker dysregulation. Such findings may change patient management strategy and result in a decision to administer an (alternative) antimicrobial regimen and/or other supportive therapies e.g. administration of an immunosuppressive agent(s). Conversely, levels of biomarkers in the biomarker signature returning to, or achieving levels comparable with, non-sepsis control levels, likely indicates an improvement in a patient's health status. Such findings may indicate that a currently administered treatment (e.g. antimicrobial agent, supportive therapies such as an immunosuppressive agent) is proving effective. The method could be implemented at regular time periods e.g. at least once an hour, once every two hours, once every six hours, to monitor a patient.

According to a third aspect, the invention provides a method for analysing biological samples, obtained from a patient predicted or diagnosed as having sepsis, to monitor the responsiveness of the patient to treatment with an antimicrobial agent(s) and/or immunosuppressive agent(s), the method comprising the steps of:
a. determining in a sample, obtained from the patient at a plurality of time points, individual levels of biomarkers representing a protein biomarker signature, wherein the protein biomarker signature comprises the biomarkers selected according to the first aspect; and
b. using changes in the individual levels of the biomarkers collectively, across the plurality of time points, to monitor the responsiveness of a patient to treatment with an antimicrobial agent(s) and/or immunosuppressive agent(s).

This aspect is particularly beneficial in identifying when a course of antimicrobial agent(s) and/or immunosuppressive agent(s), administered by a clinician, may be ineffective, or indeed effective, in terms of eradicating the causative agent of sepsis. For example, observing continued/increasing changes in the biomarkers comprising the biomarker signature likely indicates that a patient is still, or increasingly, septic, suffering organ dysfunction and/or hosting an infective agent as reflected by continued host biomarker dysregulation, as a consequence of a non-optimal antimicrobial and/or immunosuppressive regimen being administered. This scenario may be particularly relevant when a positive identification of a causative agent and/or its levels of antimicrobial susceptibility are yet to be reported by a pathology laboratory. Applying this particular method may aid the decision to administer an alternative antimicrobial and/or immunosuppressive regime. Conversely, levels of biomarkers in the biomarker signature returning to, or achieving levels comparable with, non-sepsis control levels, likely indicates an improvement in a patient's health status. Such findings may indicate that a currently administered treatment is proving effective. The method could be implemented at regular time periods e.g. at least once an hour, once every two hours, once every six hours, to monitor the responsiveness of the patient to treatment with an antimicrobial agent(s) and/or immunosuppressive agent(s).

According to a fourth aspect, the invention provides a method for selecting a therapeutic agent(s) and/or immunosuppressive agent(s) for administration to a patient predicted or diagnosed as having sepsis, the method comprising the steps of:
a. determining in a sample, obtained from the patient at a time point or plurality of time points, individual levels of biomarkers representing a protein biomarker signature, wherein the protein biomarker signature comprises the biomarkers selected according to the first aspect; and
b. using the individual levels of the biomarkers, or the changes in the individual levels of the biomarkers collectively across the plurality of time points, to select a therapeutic agent(s) and/or immunosuppressive agent(s).

This aspect is particularly beneficial in identifying an antimicrobial agent(s) and/or immunosuppressive agent(s) for administration by a clinician to a patient for the purpose of eradicating the causative agent of sepsis. For example, observing certain biomarker levels in a sample, or continued/increasing changes in the biomarkers comprising the biomarker signature in samples taken at a plurality of time points, may help inform the selection of certain antimicrobial agent(s) and/or immunosuppressive agents(s). This scenario may be particularly relevant when a positive identification of a causative agent and/or its levels of antimicrobial susceptibility are yet to be reported by a pathology laboratory. Applying this particular method may aid the decision to administer a certain antimicrobial and/or immunosuppressive regime. Conversely, levels of biomarkers in the biomarker signature returning to, or achieving levels comparable with, non-sepsis control levels, likely indicates an improvement in a patient's health status. Such findings may indicate that a currently administered treatment is proving effective. The method could be implemented at regular time periods e.g. at least once an hour, once every two hours, once every six hours, to inform on the identification of an antimicrobial agent(s) and/or immunosuppressive agent(s).

According to a fifth aspect, the invention provides use of protein biomarkers representing a protein biomarker signature for sepsis, wherein the protein biomarker signature comprises the biomarkers selected according to the first aspect.

According to a sixth aspect, the invention provides a kit for implementing at least step a) of the first aspect, second aspect, third aspect or fourth aspect, wherein the kit comprises a labelled reagent or a plurality of labelled reagents for detecting individual levels of each protein biomarker in a protein biomarker signature, in at least one sample taken from the patient, wherein the labelled reagent or reagents is/are capable of binding specifically to each protein biomarker selected according to the first aspect.

The term ‘labelled reagent’ may refer to an element capable of specifically binding to at least one of the proteins in a protein biomarker signature according to the present invention, wherein the element may be linked or associated with a labelling means that allows for identification of the presence of the protein. It is envisaged that for a given protein biomarker signature of the invention, the kit provides a plurality of elements, each of which is specific for one of the protein biomarkers in the protein biomarker signature. During use of the kit by a user, a binding event between such an element and its target protein is determined by detecting the labelling means.

The element(s) may be a biomolecule such as a protein, capable of binding to at least a region (i.e. a particular sequence or epitope) of its intended target protein of the biomarker signature.

Preferably, the labelled reagent(s) is/are antibody-based. Further preferably, the labelled reagent(s) is/are based on monoclonal antibodies. Antibodies are well established as capture means for target proteins, and can be reliably produced using known methodologies for inclusion in kits or systems. Furthermore, procedures exist for labelling antibodies with means capable of detection.

The labelling of the reagents can be achieved by a variety of ways as would be understood by the skilled person. For example, fluorescent, chromogenic, coloured or magnetic labels can be used. One common approach is the use of conjugated gold, carbon or coloured latex nanoparticles, which allow visualisation of binding/capture events between the labelled reagent and a target protein analyte. Alternatively, fluorescent or magnetic labels can require the use of a specific detector to assess whether binding events have taken place based on wavelength or magnetic signal respectively. Such detection means, whether visual or otherwise, are typically capable of quantitative measurement based on the intensity of the label. Measuring the intensity, for example by a specific visual reader, such as a camera or reader, or a non-visual detector in the case of magnetic-based labels, can enable the conversion of the label intensity into a corresponding protein analyte level or concentration. Thus, in the case of the invention, the label intensity specific for each protein biomarker determines the individual level of each protein biomarker in the protein biomarker signature, in a sample, which in turn provides an assessment of infection and/or organ dysfunction and/or sepsis.

Preferably, the reagents are labelled with gold nanoshells. Gold nanoshells consist of a 120 nm silica core coated with a 15 nm thick gold shell, and are capable of providing an increase in sensitivity relative to gold nanoparticles e.g. a 20 times increased in sensitivity. Due to the plasmon resonance of gold nanoshells, an intense blue line will be visible on white lateral flow test strips.

Preferably, the kit according to the sixth aspect further comprises a test element to which the labelled reagents are, or are capable of being, incorporated or applied.

Preferably, the test element is a lateral flow device (LFD). Such devices are well known in the art and are in particular capable of detecting the presence of an analyte(s) in a sample, such as protein biomarkers of a protein biomarker signature in a biological sample. LFDs are well suited as point-of-care devices due to their speed of testing, versatility and ease-of-use, requiring little in the way of specialist users or complex training.

LFDs comprise a membrane strip to which can be applied a liquid sample potentially containing a protein analyte(s) of interest. Upon applying a biological sample to the device, the sample flows along the membrane and encounters labelled reagent(s) (e.g. an antibody conjugate) specific for a protein analyte(s) of interest. If the protein analyte(s) is present in the sample, binding occurs between the protein analyte(s) and the labelled reagent(s), followed by further migration of the co-associated analyte(s)-labelled-reagent(s) along the membrane. A test line containing a capture reagent(s) with affinity for the target protein analyte(s) (e.g. the same antibody or antibodies but without the labelling) captures the co-associated analyte(s)-labelled-reagent(s) i.e. in a manner akin to a ‘sandwich’ assay. The labelling means associated with the migrated reagent(s) provides a detectable output. For example, a visual line is formed in the case of gold particles, thus confirming the presence of the target protein analyte(s). LFDs additionally also include a control line that confirms the sample has passed along the membrane, and that the labelled reagent(s) are active.

In the case of the invention, the protein analytes in question are in particular the biomarkers comprising the biomarker signature identified from the 22-protein panel of Table 1. To measure the biomarkers of the biomarker signature, each LFD may be split into a plurality of strips (or lanes) to accommodate the number of markers i.e. a multiplex assay. For example, each LFD assay may be split into four strips, such that ten biomarkers could be accommodated by three LFD assays. Utilising labels with different output wavelengths would enable measuring the level of each biomarkers on a multiplexed LFD assay.

The skilled person would understand that alternative approaches could be applied to the kit of the sixth aspect, in particular alternative sandwich- or competitive-based approaches. If the protein analyte(s) is present in the sample, binding may occur between the protein analyte(s) and a capture reagent(s), such as an antibody or antibodies. Following capture, labelled reagent(s) (e.g. the same antibody or antibodies but with labelling) also capable of binding to the protein analyte(s) could be applied, wherein the labelling means provides a detectable output, thus confirming the presence of the target protein analyte(s).

Alternatively, the test element is a protein array. Further alternatively, the kit in an ELISA-based approach. Both approaches employ reagents immobilised to a surface, wherein the static reagents are capable of capturing a target protein analyte(s). Further labelled reagent can be bound to the captured target protein analyte(s), ensuring a detectable (i.e. quantitative) output can be analysed to confirm the present/level of protein analyte(s) in a sample.

Other elements of the kit could be provided as required by a user. For example, the LFD may comprise a filter to ensure particulate material does not block the LFD membrane. In the case of a blood sample from a patient, the filter may remove red blood cells such that patient serum can be interrogated to measure the level of each biomarker comprising a biomarker signature to assess for infection and/or organ dysfunction and/or sepsis. The kit may further comprise a detector or reader capable of providing a quantitative measurement of the level of biomarkers in the biomarker signature.

Preferably, the kit further comprises an anticoagulant. This ensures that a blood sample taken from a patient does not clot, potentially interfering with the level of biomarkers comprising a biomarker signature that may be present in the blood sample.

According to a seventh aspect, the invention provides a system for implementing the first aspect, second aspect, third aspect or fourth aspect, the system comprising:
a. the kit of the sixth aspect;
b. a detector for monitoring, measuring or detecting the individual levels of the protein biomarkers; and
c. a computer processor configured to analyse data produced by the detector.

Operation of the system by a user can provide an output in relation to predicting, diagnosing or monitoring sepsis in a patient, or the responsiveness of the patient to treatment with an antimicrobial agent(s) and/or immunosuppressive agent(s), or selecting a therapeutic agent(s) and/or immunosuppressive agent(s) for treatment of sepsis.

The system of the seventh aspect may be computer-implemented to determine individual levels of biomarkers, representing a biomarker signature, in a sample. This would be particularly advantageous if such biomarker signature analysis is increasingly complex due to measuring a plurality of samples from a patient (i.e. taken at a plurality of time points), or measuring a plurality of samples taken from different patients. Such a computer-implemented system could enable a positive or negative readout in terms of whether infection, and/or organ dysfunction, and/or sepsis is likely to develop (or worsen/lessen). The kit or system could at least provide an indication of the likelihood of sepsis developing.

The invention also provides a method according to the first aspect, second aspect, third aspect and fourth aspect, a kit according to the sixth aspect, or system according to the seventh aspect, wherein the patient is a post-surgical patient, an immunocompromised individual, an intensive-care patient or a burn patient.

Any feature in one aspect of the invention may be applied to any other aspects of the invention, in any appropriate combination. In particular, method aspects may be applied to use, kit and system aspects and vice versa. The invention extends to methods, uses, kits or system substantially as herein described, with reference to the Examples. Furthermore, it is to be understood that the methods, kit or system aspects may include control elements (e.g. control biomarkers and respective control labelled reagents) to help validate the output of said methods, kit or system.

In all aspects, the invention may comprise, consist essentially of, or consist of any feature or combination of features.

The present invention will now be described, with reference to the following non-limiting examples and Figures, in which:

### BRIEF DESCRIPTION OF THE FIGURES

FIG. 1 is an illustration depicting the rationale for sample selection, including the selection of control samples, and the matching with sepsis patient samples;

FIG. 2 is a graph of the proportion of samples within the sepsis group outside the 90% quantiles of the SIRS and comparator groups for each of 718 protein analytes across time points; and

FIG. 3 is a dendrogram of the relatedness of protein analytes by cluster analysis.

### DETAILED DESCRIPTION

The invention provides a method for analysing a biological sample, obtained from a patient, to assess the patient for sepsis, the method comprising the steps of:
a. determining in the biological sample individual levels of biomarkers representing a protein biomarker signature; and
b. using the individual levels of the biomarkers collectively to assess the patient by predicting or diagnosing sepsis, wherein the biomarkers of the protein biomarker signature comprises at least four biomarkers from a list consisting of CCL-16, CD28, CD244, FGF21, GALNT3, GT, IL-18BP, JAM-A, LDL-R, LILRB5, LTBR, MCP-2, MMP-1, NUCB2, SIGLEC10, TNF-R1, TNF-R2, TNFRSF10A, TNFRSF11A, TNFRSF14, TRAILR2 and UPAR.

Various investigations have been carried out, as described below, to determine the predicative accuracy of a series of protein biomarker signatures to predict sepsis, according to the Sepsis-3 definition, at time points that include the day prior to (Day −1) and day of (Day 0) sepsis diagnosis.

Methods

Study Inclusion Criteria

The study recruited 4385 elective surgery patients. Patients were admitted to the study if they gave informed consent, were between 18 and 80 years of age and undergoing a procedure that, in the clinician's opinion, had a risk of causing infection and ultimately sepsis. Typically, these were abdominal and thoracic surgeries. However, other surgical procedures were permitted and included, such as an extensive maxillofacial procedure that resulted in sepsis in one case. Patients were excluded if they were either pregnant, infected with a known pathogen (HIV, Hepatitis A, B or C), immunosuppressed or withdrew consent to take part in the study at any time during their stay. All patients received the normal standard of care once enrolled.

Acquisition and Storage of Patient Samples

Blood samples were collected according to an ethically-approved protocol. Briefly, a 4 ml aliquot of patient blood was separately collected into a sterile serum separation tube. Following centrifugation, the serum was pipetted into an appropriately sized vial. All samples were then stored at −20° C. and eventually transported on dry ice. Blood collection occurred once between 1 and 7 days before surgery and then once daily on each day post-surgery. Post-operative blood collection was stopped after the patient was discharged from hospital, or after 7 days post-surgery, or once the clinician had confirmed sepsis. Additional patient information (e.g. daily patient metrics, type of surgery and microbiology results) was captured using a bespoke database provided by ItemTracker, UK. All samples collected from patients were stored at Dstl in suitably alarmed freezers that were monitored daily.

Clinical Adjudication

A Clinical Advisory Panel (CAP), comprising experts from across the UK and Germany, was tasked to provide a definitive judgement on whether a patient had developed sepsis according to the Sepsis-2 criteria. Using a blinded elicitation approach, all relevant patient data was presented to them and a silent vote was conducted. The results of this process were captured by a facilitator whose role was to ensure that no conferring had occurred and record the clinical opinion. If a consensus of opinion for a sepsis patient was achieved, then the clinicians were asked to indicate the day of sepsis diagnosis (without conferring). If consensus was again achieved then the facilitator moved to the next patient. If no consensus was reached, either for patient outcome or on day of sepsis diagnosis, then clinicians were allowed to discuss the reasons for their mixed opinions. Following a discussion, the clinicians were asked to re-vote. Key points from the discussion as well as subsequent voting that led to a consensus of opinion or a majority opinion was recorded by the facilitator for both patient classification as well as day of diagnosis for sepsis patients. It should be noted that the order of voting was sometimes randomized to mitigate the effect of peer pressure by key clinicians. Voting data was analysed using Kappa statistics to quantify the level of agreement achieved by the CAP. It was anticipated that a high level of agreement by a panel of clinical experts would give high confidence in patient classification and subsequent biomarker selection. For this study the level of agreement reached for patient classification was high.

Further analysis was undertaken to understand what proportion of the sepsis patient cohort chosen by the CAP using Sepsis-2 criteria conformed to the new Sepsis-3 definition. The former relies on the presence of SIRS caused by a microbial agent. The latter relies on organ dysfunction, as measured by changes in the SOFA score (>2), to indicate a “bad infection” that is associated with organ dysfunction.

Following clinical adjudication, 155 elective surgery patients were judged to have developed sepsis, defined according to the Sepsis-2 definition, during the study. The incidence of sepsis in the patient cohort was therefore 3.53%. Of this Sepsis-2 cohort, 98 patients were judged to have fulfilled the Sepsis-3 criteria. For all Sepsis-2 (only) and Sepsis-3 patients, age/sex/procedure-matched comparators from the cohort of patients that either developed SIRS or who had an unremarkable recovery were selected.

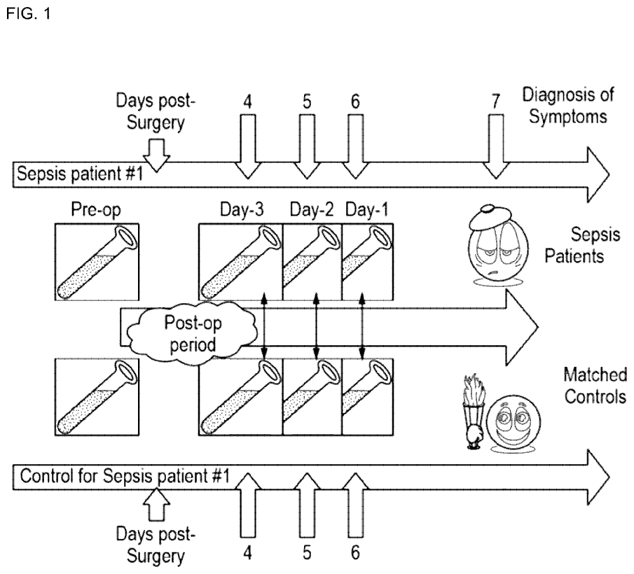

The rationale for comparator selection is illustrated in FIG. 1, along with which patient samples were analysed and how the timeframes for patient samples taken at different days post-surgery were standardized. The time course of the development of sepsis in a patient is indicated by the Sepsis patient #1 bar. From the large number of patients who did not go on to develop sepsis following surgery, a suitable age/sex/procedure-matched control is identified and used as a comparator. In this example, the day of diagnosis of sepsis is day 7 post-infection. Therefore, the 3 days before sepsis diagnosis are days 4, 5 and 6 post-surgery. In terms of pre-symptomatic diagnosis, this may also be noted as Days −3, −2 and −1. In order to provide a robust and relevant post-operative comparison for each of the 3 days before sepsis diagnosis, the equivalent post-operative blood sample from the age/gender/procedure-matched comparator was used. In this case, the blood samples taken from days 4, 5 and 6 post surgery were used for comparison, acting as Day −3, −2 and −1 controls. The process of matching the pre-symptomatic blood samples of patients who went on to develop sepsis with their most appropriate post-operative comparators was then repeated for all sepsis patients. Table 2 summarises a series of top-level characteristics for patients involved in the study.

TABLE 2
Summary of patient ages, gender, delay
for sepsis and types of surgery

| | Sepsis | Controls | SIRS |
|---|---|---|---|
| | n = 50 | n = 50 | n = 49 |
| Median | 65 | 64.5 | 66 |
| age (IQR) | (58.5-74) | (56.75-73) | (54.5-73) |
| Gender | 7/43 | 5/45 | 6/43 |
| (female/male) | | | |
| Median day of | 3 | n/a | n/a |
| sepsis diagnosis | (2-4) | | |
| (IQR) | | | |
| Median SOFA | 4 | 0 | 0 |
| score on day of | (0-7) | (0-0) | (0-0) |
| sepsis diagnosis | | | |
| (or equivalent | | | |
| day post-surgery) | | | |

O-Link Analysis

Analysis of patient proteome was conducted on samples from 50 patients who went on to develop sepsis using the O-link array platform (O-link Proteomics, Uppsala, Sweden). Additional samples from age/gender/procedure-matched control patients (n=50) and patients who developed SIRS (n=49) were also used. Analysis was performed in accordance with manufacturer's instructions. The panel chips used included: CARDIOMETABOLIC (v.3603), CARDIOVASCULAR II (v.5004), CARDIOVASCULAR III (v.6112), CELL REGULATION (v.3701), IMMUNE RESPONSE (v.3203), INFLAMMATION (v.3012), METABOLISM (v.3402) and ORGAN DAMAGE (v.3311).

Data Analysis

Graphs were generated using the software Graphpad PRISM V8.0. Statistical analysis was performed using IBM SPSS V26.0. NPX data from the three panels were collated into a single file. Where proteins had been investigated in more than one panel, the mean value was taken. Some missing data was replaced using a regression based with random effect method of imputation. These missing values principally consisted of one sample in certain analytes. All data NPX was used regardless of whether the values were within the limits of quantification or whether all quality controls were passed.

Data was organised into groups (‘SIRS’; ‘Sepsis’; and ‘Control’) and time prior to diagnosis of condition (‘Baseline’; ‘Time of Conventional Diagnosis’; ‘1 Day Prior to Diagnosis’; 2 Days Prior to Diagnosis’; and ‘3 Days Prior to diagnosis’). The 95^{th} and 5^{th} percentile were estimated for each protein analyte, for each time point, for the control and SIRS group combined. The proportion of proteins that were outside these percentages were calculated for each time point.

The top 40 protein analytes at time of diagnosis and 1 day prior were selected (i.e. the protein analytes outside the 90% quantiles of the SIRS and control samples in the most ‘sepsis’ samples. These protein analytes were subjected to stepwise cluster using Pearson's correlations. A dendrogram was then used to select protein analytes that were most unrelated. The “left-most” members of each cluster at different levels of similarity were selected because these represented the least related protein to the next cluster. The ability of different groups of protein analytes to predict sepsis was assessed using multilayer perceptron neutral networks. (Other algorithms that can manage heterogeneity, such as random forests are also suitable. Conversely, linear discriminant analysis would be less useful for the same reason). The SPSS adaptive algorithm was used to fine-tune the methodology of each analysis. The neural nets were trained ten times using 70% of the data at both time of diagnosis and 1 day prior. The same 70% of individuals was used at both time points. For each of the iterations, a random selection program was generated that ensured that the same 70% was used at both time points. The other 30% and other time points were used to predict efficacy. Efficacy was estimated and compared by Receiver Operator Characteristics (ROC) analysis of the membership estimates and the AUC of the ROC curve.

Results

Assay Reliability

In order to consider the general reliability of the O-link assay system, single analyte measurements were plotted onto scatter plots. These plots included 20 analytes that had been measured twice and two analytes that had been measured three times. A very high level of correlation was found in these data sets. Level of correlation typically corresponded to where the range of values was greatest. This analysis also allowed an estimation of the likely rate with which outliers occur. A total of 18 obvious outliers was observed in 28,106 readings indicating failure rate of 0.064% (0.041%, 0.101% 95% confidence intervals using the Wilson-Brown method).

Down-Selection of Protein Analytes Based on Likely Usability

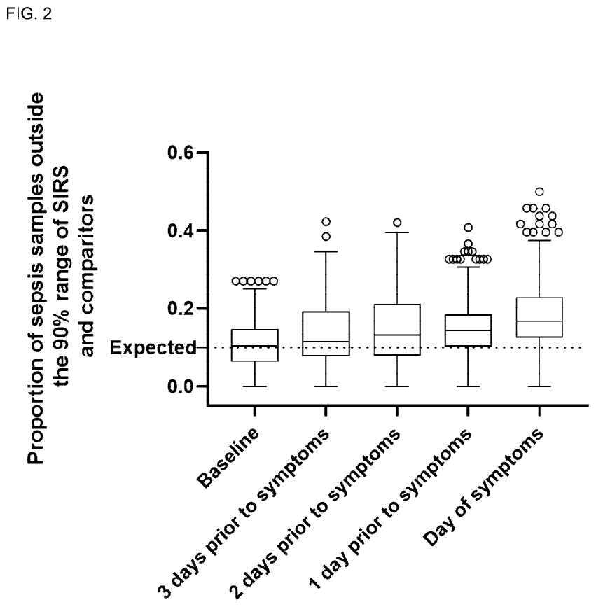

The O-link output generated data for 718 protein analytes. A metric was needed for rapid down-selection of target protein analytes where the greatest proportion of readings in the sepsis group were outside the normal range of the two control data sets (comparator controls and SIRS). The strategy devised included first calculating the 5^{th} and 95^{th} percentiles of the two control groups at each time point and then using logic functions to numerate the number of sepsis readings at the same time point that were outside this range. The greatest number of sepsis samples with specific proteins outside this 90% range were considered most likely to be useful in sepsis diagnosis. The frequency of protein analytes meeting this metric was found to increase at time points closer to diagnosis (FIG. 2; data expressed as a Turkey plot, where protein analytes outside the 75% quartile+1.5× the IQR are expressed as symbols). This is consistent with expectations, as the individual's biology becomes more dysregulated by the sepsis.

Various down-selections based on this devised metric were made, identifying 40 protein analytes with the greatest values for these metrics at day of diagnosis and day prior to diagnosis. There was significant overlap in these protein analyte sets, providing 54 unique protein analytes.

Selection of Protein Analytes to Provide the Best Complementary Benefit

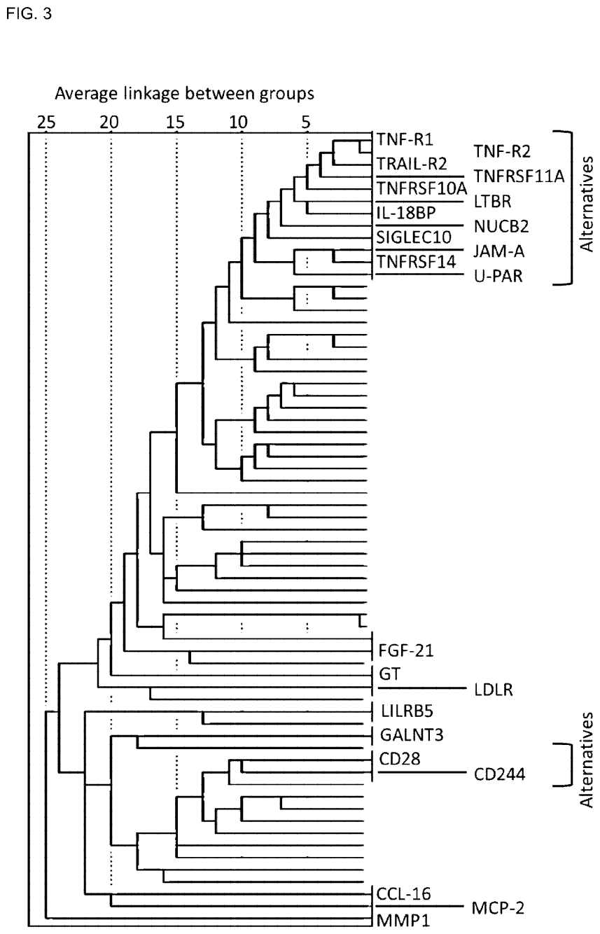

Further down-selection of the 54 candidate protein analytes was based on reasoning that the best approach would be to consider the protein analytes whose expression correlated least well to each other. To this end, cluster analysis of the protein analytes was performed using Pearson's correlations. This analysis generated a dendrogram of comparative relatedness (FIG. 3). Using a relatedness threshold of about between 18 and 19 Average Linkage provided ten clusters. Representatives that are “far right” were selected, as this will be the least related to the next cluster. Two of these clusters contain multiple closely related protein analytes that might be used as representatives.

Protein biomarker signatures containing between four and ten proteins showed evidence for predictive power when visualised individually. Importantly, the proteins within a biomarker signature correlated with each other very poorly. In this respect, it was reasonable to assume that these protein analytes will complement each other well in a multiple protein analyte diagnostic. Tumour Necrosis Factor Receptor 1 (TNF-R1) was part of a large cluster. In this respect, alternative protein analytes might be used with little effect and the fact that these alternatives are found in similar concentrations can also be visualised. Similarly, CD28 is similarly expressed to CD244.

Evaluation of Possible Predictive Power of Protein Analyte Panels

In order to evaluate the predictive power of these panels of proteins, multilayer perceptron neuronal networks were used. Given that sepsis is a blanket term for a variety of infections with pathologies, it was postulated that the best tool for diagnosis would be non-linear. The SPSS adaptive algorithm was used to fine-tune the numbers of nodes and methods. Training sets (70%) were selected randomly using Microsoft Excel random number generator and bespoke generated work sheets that allowed consistency across time points. The same 10 training/test sets were run for each iteration of analysis.

It was found that representatives (n=22) of the ten clusters at ˜20 similarity gave good ROC curves at day of and day prior to diagnosis. Table 3 describes the predictive efficacy, described in terms of AUC, of a series of biomarker subsets produced from the list of 22 proteins down-selected for the biomarker signature (SD: standard deviation).

TABLE 3
Predictive efficacy for sepsis of a series of biomarker signatures derived from 22 down-selected proteins

| | | Day −1 | | | | | Day 0 | | | | |
|---|---|---|---|---|---|---|---|---|---|---|---|
| Ref | Biomarkers | Mean | Median | SD | 25^{th} Q | 75^{th} Q | Mean | Median | SD | 25^{th} Q | 75^{th} Q |
| A | CCL16, CD28, FGF21, | 0.80 | 0.82 | 0.07 | 0.75 | 0.86 | 0.86 | 0.86 | 0.07 | 0.85 | 0.91 |
| | GALNT3, GT, LDL-R, | | | | | | | | | | |
| | LILRB5, MCP-2, MMP-1, | | | | | | | | | | |
| | TNF-R1 | | | | | | | | | | |
| B | CCL16, CD28, GALNT3, | 0.77 | 0.79 | 0.03 | 0.75 | 0.80 | 0.83 | 0.80 | 0.04 | 0.80 | 0.85 |
| | GT, LDL-R, LILRB5, | | | | | | | | | | |
| | MCP-2, MMP-1, TNF-R1 | | | | | | | | | | |
| C | CCL16, CD28, GALNT3, | 0.75 | 0.75 | 0.03 | 0.74 | 0.77 | 0.82 | 0.78 | 0.04 | 0.79 | 0.86 |
| | GT, LDL-R, LILRB5, | | | | | | | | | | |
| | MMP-1, TNF-R1 | | | | | | | | | | |
| D | CCL16, GALNT3, GT, | 0.74 | 0.76 | 0.06 | 0.75 | 0.78 | 0.76 | 0.76 | 0.06 | 0.75 | 0.79 |
| | LDL-R, LILRB5, MMP-1, | | | | | | | | | | |
| | TNF-R1 | | | | | | | | | | |
| E | CCL16, GALNT3, LDL-R, | 0.75 | 0.75 | 0.04 | 0.71 | 0.78 | 0.79 | 0.77 | 0.05 | 0.76 | 0.84 |
| | LILRB5, MMP-1, TNF-R1 | | | | | | | | | | |
| F | CCL16, GALNT3, LILRB5, | 0.73 | 0.73 | 0.03 | 0.71 | 0.75 | 0.78 | 0.72 | 0.05 | 0.73 | 0.80 |
| | MMP-1, TNF-R1 | | | | | | | | | | |
| G | CCL16, LILRB5, MMP-1, | 0.76 | 0.76 | 0.03 | 0.75 | 0.78 | 0.80 | 0.78 | 0.03 | 0.80 | 0.82 |
| | TNF-R1 | | | | | | | | | | |
| H | LILRB5, MMP-1, TNF-R1 | 0.73 | 0.73 | 0.02 | 0.71 | 0.74 | 0.74 | 0.74 | 0.02 | 0.73 | 0.76 |
| I | MMP-1, TNF-R1 | 0.70 | 0.70 | 0.02 | 0.69 | 0.70 | 0.73 | 0.71 | 0.02 | 0.71 | 0.74 |
| J | CCL16, CD28, FGF21, | 0.76 | 0.77 | 0.03 | 0.74 | 0.79 | 0.87 | 0.86 | 0.04 | 0.85 | 0.90 |
| | MCP-2, LTBR | | | | | | | | | | |
| K | CCL16, CD244, FGF21, | 0.81 | 0.82 | 0.04 | 0.78 | 0.83 | 0.87 | 0.86 | 0.02 | 0.86 | 0.89 |
| | GALNT3, GT, LDL-R, | | | | | | | | | | |
| | LILRB5, MCP-2, MMP-1, | | | | | | | | | | |
| | TNF-R1 | | | | | | | | | | |
| L | CCL16, CD28, FGF21, | 0.78 | 0.79 | 0.07 | 0.75 | 0.82 | 0.84 | 0.86 | 0.09 | 0.83 | 0.90 |
| | GALNT3, GT, LDL-R, | | | | | | | | | | |
| | LILRB5, MCP-2, MMP-1, | | | | | | | | | | |
| | TNF-R2 | | | | | | | | | | |
| M | CCL16, CD28, FGF21, | 0.78 | 0.80 | 0.06 | 0.74 | 0.82 | 0.85 | 0.84 | 0.04 | 0.83 | 0.87 |
| | GALNT3, GT, LDL-R, | | | | | | | | | | |
| | LILRB5, MCP-2, MMP-1, | | | | | | | | | | |
| | TRAIL-R2 | | | | | | | | | | |
| N | CCL16, CD28, FGF21, | 0.86 | 0.88 | 0.06 | 0.86 | 0.91 | 0.86 | 0.87 | 0.06 | 0.86 | 0.90 |
| | GALNT3, GT, LDL-R, | | | | | | | | | | |
| | LILRB5, MCP-2, MMP-1, | | | | | | | | | | |
| | TNFRSF11A | | | | | | | | | | |
| O | CCL16, CD28, FGF21, | 0.77 | 0.76 | 0.05 | 0.73 | 0.77 | 0.85 | 0.82 | 0.05 | 0.82 | 0.88 |
| | GALNT3, GT, LDL-R, | | | | | | | | | | |
| | LILRB5, MCP-2, MMP-1, | | | | | | | | | | |
| | TNFRSF10A | | | | | | | | | | |
| P | CCL16, CD28, FGF21, | 0.79 | 0.78 | 0.08 | 0.71 | 0.84 | 0.85 | 0.84 | 0.05 | 0.81 | 0.88 |
| | GALNT3, GT, LDL-R, | | | | | | | | | | |
| | LILRB5, MCP-2, MMP-1, | | | | | | | | | | |
| | LTBR | | | | | | | | | | |
| Q | CCL16, CD28, FGF21, | 0.76 | 0.76 | 0.06 | 0.71 | 0.81 | 0.82 | 0.78 | 0.09 | 0.76 | 0.89 |
| | GALNT3, GT, LDL-R, | | | | | | | | | | |
| | LILRB5, MCP-2, MMP-1, | | | | | | | | | | |
| | IL-18BP | | | | | | | | | | |
| R | CCL16, CD28, FGF21, | 0.76 | 0.76 | 0.06 | 0.71 | 0.81 | 0.82 | 0.78 | 0.09 | 0.76 | 0.89 |
| | GALNT3, GT, LDL-R, | | | | | | | | | | |
| | LILRB5, MCP-2, MMP-1, | | | | | | | | | | |
| | NUCB2 | | | | | | | | | | |
| S | CCL16, CD28, FGF21, | 0.76 | 0.74 | 0.07 | 0.70 | 0.81 | 0.84 | 0.78 | 0.06 | 0.80 | 0.87 |
| | GALNT3, GT, LDL-R, | | | | | | | | | | |
| | LILRB5, MCP-2, MMP-1, | | | | | | | | | | |
| | SIGLEC10 | | | | | | | | | | |
| T | CCL16, CD28, FGF21, | 0.72 | 0.70 | 0.07 | 0.67 | 0.76 | 0.81 | 0.78 | 0.06 | 0.77 | 0.85 |
| | GALNT3, GT, LDL-R, | | | | | | | | | | |
| | LILRB5, MCP-2, MMP-1, | | | | | | | | | | |
| | JAM-A | | | | | | | | | | |
| U | CCL16, CD28, FGF21, | 0.76 | 0.77 | 0.07 | 0.71 | 0.82 | 0.84 | 0.83 | 0.05 | 0.83 | 0.87 |
| | GALNT3, GT, LDL-R, | | | | | | | | | | |
| | LILRB5, MCP-2, MMP-1, | | | | | | | | | | |
| | TNFRSF14 | | | | | | | | | | |
| V | CCL16, CD28, FGF21, | 0.80 | 0.81 | 0.06 | 0.75 | 0.84 | 0.86 | 0.85 | 0.05 | 0.80 | 0.90 |
| | GALNT3, GT, LDL-R, | | | | | | | | | | |
| | LILRB5, MCP-2, MMP-1, | | | | | | | | | | |
| | U-PAR | | | | | | | | | | |

It will be understood that the present invention has been described above purely by way of example, and modification of detail can be made within the scope of the invention. For example, alternative approaches to a ‘sandwich’ reaction may be considered in a method, kit or system e.g. competitive assay, providing that such approaches enable quantitative measurement of the individual biomarkers of the biomarker signature in the sample being analysed. The labelled reagent(s) may include element(s) capable of specifically binding to at least one of the proteins in a protein biomarker signature according to the present invention, wherein the element(s) may be capable of being linked or associated with a labelling means during application of the method, kit or system that allows for identification of the presence of the protein. Each feature disclosed in the description and (where appropriate) the claims may be provided independently or in any appropriate combination.

Moreover, the invention has been described with specific reference to methods and associated uses, kits and systems relating to assessing sepsis defined according to the Sepsis-3 definition. Additional applications of the invention will occur to the skilled person.

1. A method for analyzing a biological sample, obtained from a patient, to assess whether the patient may develop sepsis or to diagnose the patient as having sepsis, the method comprising the steps of:
a. determining in the biological sample individual levels of protein biomarkers representing a protein biomarker signature; and,
b. using the individual levels of the protein biomarkers collectively to assess whether the patient may develop sepsis or to diagnose a patient as having sepsis,
wherein the protein biomarkers of the protein biomarker signature comprise at least four of CCL-16, CD28, CD244, FGF21, GALNT3, GT, IL-18BP, JAM-A, LDL-R, LILRB5, LTBR, MCP-2, MMP-1, NUCB2, SIGLEC10, TNF-R1, TNF-R2, TNFRSF10A, TNFRSF11A, TNFRSF14, TRAILR2 and UPAR.

2. The method according to claim 1, wherein the protein biomarker signature comprises CCL-16 and MCP-2.

3. The method according to claim 2, wherein the protein biomarker signature consists of LTBR, CCL-16, CD28, FGF21 and MCP-2.

4. The method according to claim 2, wherein the protein biomarker signature further comprises GALNT3, GT, LDL-R, LILRB5 and MMP-1.

5. The method according to claim 4, wherein the protein biomarker signature further comprises FGF21.

6. The method according to claim 5, wherein the protein biomarker signature consists of CCL16, CD28, FGF21, GALNT3, GT, LDL-R, LILRB5, MCP-2, MMP-1 and TNFRSF11A.

7. The method according to claim 5, wherein the protein biomarker signature consists of CCL16, CD244, FGF21, GALNT3, GT, LDL-R, LILRB5, MCP-2, MMP-1 and TNF-R1.

8. The method according to claim 5, wherein protein biomarker signature consists of CCL16, CD28, FGF21, GALNT3, GT, LDL-R, LILRB5, MCP-2, MMP-1 and TNF-R1.

9. The method according to claim 5, wherein the protein biomarker signature consists of CCL16, CD28, FGF21, GALNT3, GT, LDL-R, LILRB5, MCP-2, MMP-1 and one of U-PAR or TRAIL-R2.

10. (canceled)

11. The method according to claim 1, wherein the protein biomarker signature further comprises at least one additional biomarker from a list consisting of PCT, lactate, CRP, D-Dimer and PSP.

12. A method for analyzing biological samples, obtained from a patient at risk of, or having developed, sepsis, to monitor the patient, the method comprising the steps of:
a. determining in the biological samples, obtained from the patient at a plurality of time points, individual levels of protein biomarkers representing a protein biomarker signature, wherein the protein biomarker signature comprises the biomarkers selected according to the method of claim 1; and
b. using changes in the individual levels of the protein biomarkers collectively, across the plurality of time points, to monitor the patient and to predict whether the patient may develop sepsis, or to monitor the progression of sepsis in the patient.

13. A method for analyzing biological samples, obtained from a patient predicted or diagnosed as having sepsis, to monitor the responsiveness of the patient to treatment with an antimicrobial agent and/or immunosuppressive agent, the method comprising the steps of:
a. determining in a sample, obtained from the patient at a plurality of time points, individual levels of biomarkers representing a protein biomarker signature, wherein the protein biomarker signature comprises the biomarkers selected according to the method of claim 1; and
b. using changes in the individual levels of the biomarkers collectively, across the plurality of time points, to monitor the responsiveness of a patient to treatment with the antimicrobial agent and/or immunosuppressive agent.

14. A method for selecting a therapeutic agent and/or immunosuppressive agent for administration to a patient predicted or diagnosed as having sepsis, the method comprising the steps of:
a. determining in a sample, obtained from the patient at a time point or plurality of time points, individual levels of biomarkers representing a protein biomarker signature, wherein the protein biomarker signature comprises the biomarkers selected according to the method of claim 1; and
b. using the individual levels of the biomarkers, or the changes in the individual levels of the biomarkers collectively across the plurality of time points, to select the therapeutic agent and/or immunosuppressive agent.

15. (canceled)

16. A kit for implementing at least step a) of the method of claim 1, wherein the kit comprises a labelled reagent or a plurality of labelled reagents for detecting individual levels of each protein biomarker in a protein biomarker signature, in at least one sample taken from the patient, wherein the labelled reagent or reagents is/are capable of binding specifically to each protein biomarker selected according to the method of claim 1.

17. The kit according to claim 16, wherein the labelled reagents are antibody-based.

18. The kit according to claim 16, further comprising a test element to which the labelled reagents are, or are capable of being, incorporated or applied.

19. The kit according to claim 18, wherein the test element is a lateral flow device.

20. The kit according to claim 18, wherein the test element is a protein array.

21. The kit according to claim 16, wherein the kit further comprises an anticoagulant.

22. A system comprising:
i. the kit of claim 16;
j. a detector for monitoring, measuring or detecting the individual levels of the protein biomarkers; and
k. a computer processor configured to analyse data produced by the detector.
